(19) United States
(12) Patent Application Publication
(10) Pub. No.: US 2008/0254453 A1
(43) Pub. Date: Oct. 16, 2008

(54) Analysis of methylation using selective adaptor ligation

(75) Inventors: Michael H. Shapero, Redwood City, CA (US); Shivani Nautiyal, Portola Valley, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: AFFYMETRIX, INC, Santa Clara, CA (US)

(21) Appl. No.: 11/734,372

(22) Filed: Apr. 12, 2007

(51) Int. Cl. C12Q 1/68; C12P 19/34

(52) U.S. Cl. 435/6; 435/91.1

(57) ABSTRACT

Methods of analyzing DNA to identify regions of the genome that are methylated in a genomic sample are disclosed. In one aspect genomic DNA is fragmented using a restriction enzyme with a degenerate recognition site, methylated restriction fragments are separated from unmethylated fragments by affinity purification. The complexity of the methylated fragments is reduced by amplification of a subset of the fragments using adaptors that ligate to a subset of the fragments. The amplified product is fragmented, labeled and hybridized to an array of probes. The hybridization pattern is analyzed to determine methylation status of cytosines.

### RELATED APPLICATIONS

The present application claims priority to U.S. Provisional Application No. 60/774,705, filed Apr. 12, 2006, the entire disclosure of which is incorporated herein by reference.

### FIELD OF THE INVENTION

The present invention relates to arrays and methods for detecting methylation of nucleic acids.

### BACKGROUND OF THE INVENTION

The genomes of higher eukaryotes contain the modified nucleoside 5-methyl cytosine (5-meC). This modification is usually found as part of the dinucleotide CpG. Cytosine is converted to 5-methylcytosine in a reaction that involves flipping a target cytosine out of an intact double helix and transfer of a methyl group from S-adenosylmethionine by a methyltransferase enzyme (Klimasauskas et al., Cell 76:357-369, 1994). This enzymatic conversion is the only epigenetic modification of DNA known to exist in vertebrates and is essential for normal embryonic development (Bird, Cell 70:5-8, 1992; Laird and Jaenisch, Human Mol. Genet. 3:1487-1495, 1994; and Li et al., Cell 69:915-926, 1992).

The frequency of the CpG dinucleotide in the human genome is only about 20% of the statistically expected frequency, possibly because of spontaneous deamination of 5-meC to T (Schoreret et al., Proc. Natl. Acad. Sci. USA 89:957-961, 1992). There are about 28 million CpG doublets in a haploid copy of the human genome and it is estimated that about 70-80% of the cytosines at CpGs are methylated. Regions where CpG is present at levels that are approximately the expected frequency are referred to as “CpG islands” (Bird, A. P., Nature 321:209-213, 1986). These regions have been estimated to comprise about 1% of vertebrate genomes and account for about 15% of the total number of CpG dinucleotides. CpG islands are typically between 0.2 and 1 kb in length and are often located upstream of housekeeping and tissue-specific genes. CpG islands are often located upstream of transcribed regions, but may also extend into transcribed regions. About 2-4% of cytosines are methylated and probably the majority of cytosines that are 5′ of Gs are methylated. Most of the randomly distributed CpGs are methylated, but only about 20% of the CpGs in CpG islands are methylated.

DNA methylation is an epigenetic determinant of gene expression. Patterns of CpG methylation are heritable, tissue specific, and correlate with gene expression. The consequence of methylation is usually gene silencing. DNA methylation also correlates with other cellular processes including embryonic development, chromatin structure, genomic imprinting, somatic X-chromosome inactivation in females, inhibition of transcription and transposition of foreign DNA and timing of DNA replication. When a gene is highly methylated it is less likely to be expressed, possibly because CpG methylation prevents transcription factors from recognizing their cognate binding sites. Proteins that bind methylated DNA may also recruit histone deacetylase to condense adjacent chromatin. Such “closed” chromatin structures prevent binding of transcription factors. Thus the identification of sites in the genome containing 5-meC is important in understanding cell-type specific programs of gene expression and how gene expression profiles are altered during both normal development and diseases such as cancer. Precise mapping of DNA methylation patterns in CpG islands has become essential for understanding diverse biological processes such as the regulation of imprinted genes, X chromosome inactivation, and tumor suppressor gene silencing in human cancer caused by increase methylation.

Methylation of cytosine residues in DNA plays an important role in gene regulation. Methylation of cytosine may lead to decreased gene expression by, for example, disruption of local chromatin structure, inhibition of transcription factor-DNA binding, or by recruitment of proteins which interact specifically with methylated sequences and prevent transcription factor binding. DNA methylation is required for normal embryonic development and changes in methylation are often associated with disease. Genomic imprinting, X chromosome inactivation, chromatin modification, and silencing of endogenous retroviruses all depend on establishing and maintaining proper methylation patterns. Abnormal methylation is a hallmark of cancer cells and silencing of tumor suppressor genes is thought to contribute to carcinogenesis. Methylation mapping using microarray-based approaches may be used, for example, to profile cancer cells revealing a pattern of DNA methylation that may be used, for example, to diagnose a malignancy, predict treatment outcome or monitor progression of disease. Methylation in eukaryotes can also function to inhibit the activity of viruses and transposons, see Jones et al., EMBO J. 17:6385-6393 (1998). Alterations in the normal methylation process have also been shown to be associated with genomic instability (Lengauer et al., Proc. Natl. Acad. Sci. USA 94:2545-2550, 1997). Such abnormal epigenetic changes may be found in many types of cancer and can serve as potential markers for oncogenic transformation.

### SUMMARY OF THE INVENTION

Methods for analyzing the methylation status of cytosines in genomic DNA are disclosed. In one aspect genomic DNA is fragmented with a restriction enzyme that has at least one degenerate position in the recognition site, adaptors are ligated to the fragments, methylated fragments are affinity purified and a subset of the fragments are amplified. The amplified subset is enriched relative to the genomic DNA sample for fragments that were methylated in the genomic sample. The enriched sample has a complexity that is reduced relative to the genomic sample, there are fewer different sequences present but of those fragments that are present most were methylated in the genomic sample.

Methods for determining which fragments are present in the enriched sample are disclosed and preferably include hybridization of the sample to an array of nucleic acid probes. The array may be, for example, a promoter array, a CpG island array or a tiling array.

In some aspects the affinity selection is performed after fragmentation but before adaptor ligation. In other aspects the affinity selection is performed after adaptor ligation. Amplification generally results in loss of epigenetic modifications such as methylation so the affinity selection should preferably be performed prior to amplification. Amplification is preferably primer directed using primers complementary to sequences on the adaptor or adaptors and may be by PCR.

The fragments in the enriched sample may be further fragmented and labeled, for example, with biotin using TdT. Fragmentation may be by DNaseI or by incorporation of dUTP during amplification followed by treatment with UDG to generate abasic sites. The abasic sites may be cleaved by heat, pH or treatment with an abasic endonuclease such as APE 1.

In some aspects the methods are used to classify a tissue into a class, for example, a known tumor class. The hybridization pattern obtained from the tissue sample, using the disclosed methods, is compared to hybridization patterns from samples from tissues of known tumor class, obtained using the disclosed methods.

### BRIEF DESCRIPTION OF THE DRAWINGS

FIG. 1A shows a schematic of a method of methylation analysis with adaptor ligation followed by affinity enrichment and amplification.

FIG. 1B shows a schematic similar to that shown in FIG. 1a but with the affinity enrichment step occurring prior to the adaptor ligation step.

FIG. 2 shows workflows for four different schematics for selective adaptor ligations based methylation analysis.

FIG. 3 shows a schematic of a method for selective amplification of methylated fragments.

FIG. 4 shows a schematic of a method to analyze different subsets of the genome by using different restriction enzymes.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being, but may also include other organisms including but not limited to mammals, plants, fungi, bacteria or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5 th Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. PGPub Nos. 20070065816 and 20030036069), and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with hybridization to an array, the sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675. The sample may be amplified on the array. See, for example, U.S. Pat. No. 6,300,070 which is incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245), rolling circle amplification (RCA) (for example, Fire and Xu, PNAS 92:4641 (1995) and Liu et al., J. Am. Chem. Soc. 118:1587 (1996)) and nucleic acid based sequence amplification (NABSA), (See, U.S. Pat. Nos. 5,409,818, 5,554,517, and 6,063,603). Other amplification methods that may be used are described in, U.S. Pat. Nos. 5,242,794, 5,494,810, 4,988,617 and in U.S. Ser. No. 09/854,317. Other amplification methods are also disclosed in Dahl et al., Nuc. Acids Res. 33(8):e71 (2005) and circle to circle amplification (C2CA) Dahl et al., PNAS 101:4548 (2004). Locus specific amplification and representative genome amplification methods may also be used.

Additional methods of sample preparation and techniques for reducing the complexity of a nucleic sample are described in Dong et al., Genome Research 11, 1418 (2001), in U.S. Pat. Nos. 6,872,529, 6,361,947, 6,391,592 and 6,107,023, US Patent Publication Nos. 20030096235 and 20030082543 and U.S. patent application Ser. No. 09/916,135.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. PGPub No. 20040012676 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in PGPub Nos. 20040012676 and 20050059062 and in PCT Application PCT/US99/06097 (published as WO 99/47964), each of which also is hereby incorporated by reference in its entirety for all purposes. Instruments and software may also be purchased commercially from various sources, including Affymetrix.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

Methods for detection of methylation status are disclosed, for example, in Fraga and Esteller, BioTechniques 33:632-649 (2002) and Dahl and Guldberg Biogerontology 4:233-250 (2003). Methylation detection using bisulfite modification and target specific PCR have been disclosed, for example, in U.S. Pat. Nos. 5,786,146, 6,200,756, 6,143,504, 6,265,171, 6,251,594, 6,331,393, and 6,596,493. U.S. Pat. No. 6,884,586 disclosed methods for methylation analysis using nicking agents and isothermal amplification.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. PGPub Nos. 20030097222, 20020183936, 20030100995, 20030120432, 20040002818, 20040126840, and 20040049354.

All documents, i.e., publications and patent applications, cited in this disclosure, including the foregoing, are incorporated by reference herein in their entireties for all purposes to the same extent as if each of the individual documents were specifically and individually indicated to be so incorporated by reference herein in its entirety.

b) Definitions “Adaptor sequences” or “adaptors” are generally oligonucleotides of at least 5, 10, or 15 bases and preferably no more than 50 or 60 bases in length; however, they may be even longer, up to 100 or 200 bases. Adaptor sequences may be synthesized using any methods known to those of skill in the art. For the purposes of this invention they may, as options, comprise primer binding sites, recognition sites for endonucleases, common sequences and promoters. The adaptor may be entirely or substantially double stranded or entirely single stranded. A double stranded adaptor may comprise two oligonucleotides that are at least partially complementary. The adaptor may be phosphorylated or unphosphorylated on one or both strands.

Adaptors may be more efficiently ligated to fragments if they comprise a substantially double stranded region and a short single stranded region which is complementary to the single stranded region created by digestion with a restriction enzyme. For example, when DNA is digested with the restriction enzyme EcoRI the resulting double stranded fragments are flanked at either end by the single stranded overhang 5′-AATT-3′, an adaptor that carries a single stranded overhang 5′-AATT-3′ will hybridize to the fragment through complementarity between the overhanging regions. This “sticky end” hybridization of the adaptor to the fragment may facilitate ligation of the adaptor to the fragment but blunt ended ligation is also possible. Blunt ends can be converted to sticky ends using the exonuclease activity of the Klenow fragment. For example when DNA is digested with PvuII the blunt ends can be converted to a two base pair overhang by incubating the fragments with Klenow in the presence of dTTP and dCTP. Overhangs may also be converted to blunt ends by filling in an overhang or removing an overhang.

Methods of ligation will be known to those of skill in the art and are described, for example in Sambrook et al. (2001) and the New England BioLabs catalog both of which are incorporated herein by reference for all purposes. Methods include using T4 DNA Ligase which catalyzes the formation of a phosphodiester bond between juxtaposed 5′ phosphate and 3′ hydroxyl termini in duplex DNA or RNA with blunt and sticky ends; Taq DNA Ligase which catalyzes the formation of a phosphodiester bond between juxtaposed 5′ phosphate and 3′ hydroxyl termini of two adjacent oligonucleotides which are hybridized to a complementary target DNA; E. coli DNA ligase which catalyzes the formation of a phosphodiester bond between juxtaposed 5′-phosphate and 3′-hydroxyl termini in duplex DNA containing cohesive ends; and T4 RNA ligase which catalyzes ligation of a 5′ phosphoryl-terminated nucleic acid donor to a 3′ hydroxyl-terminated nucleic acid acceptor through the formation of a 3′->5′ phosphodiester bond, substrates include single-stranded RNA and DNA as well as dinucleoside pyrophosphates; or any other methods described in the art. Fragmented DNA may be treated with one or more enzymes, for example, an endonuclease, prior to ligation of adaptors to one or both ends to facilitate ligation by generating ends that are compatible with ligation.

Adaptors may also incorporate modified nucleotides that modify the properties of the adaptor sequence. For example, phosphorothioate groups may be incorporated in one of the adaptor strands. A phosphorothioate group is a modified phosphate group with one of the oxygen atoms replaced by a sulfur atom. In a phosphorothioated oligo (often called an “S-Oligo”), some or all of the internucleotide phosphate groups are replaced by phosphorothioate groups. The modified backbone of an S-Oligo is resistant to the action of most exonucleases and endonucleases. Phosphorothioates may be incorporated between all residues of an adaptor strand, or at specified locations within a sequence. A useful option is to sulfurize only the last few residues at each end of the oligo. This results in an oligo that is resistant to exonucleases, but has a natural DNA center.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “array plate” as used herein refers to a body having a plurality of arrays in which each microarray is separated by a physical barrier resistant to the passage of liquids and forming an area or space, referred to as a well, capable of containing liquids in contact with the probe array.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa, Nucleic Acids Res. 12:203 (1984), incorporated herein by reference.

The term “epigenetic” as used herein refers to factors other than the primary sequence of the genome that affect the development or function of an organism, they can affect the phenotype of an organism without changing the genotype. Epigenetic factors include modifications in gene expression that are controlled by heritable but potentially reversible changes in DNA methylation and chromatin structure. Methylation patterns are known to correlate with gene expression and in general highly methylated sequences are poorly expressed.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na⁺], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004, available at Affymetrix.com.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490 and other nucleic acid analogs and nucleic acid mimetics.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “label” as used herein refers to a luminescent label, a light scattering label or a radioactive label. Fluorescent labels include, inter alia, the commercially available fluorescein phosphoramidites such as Fluoreprime (Pharmacia), Fluoredite (Millipore) and FAM (ABI). See U.S. Pat. No. 6,287,778.

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “nucleic acids” as used herein may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Restriction enzymes or restriction endonucleases and their properties are well known in the art. A wide variety of restriction enzymes are commercially available, from, for example, New England Biolabs. Restriction enzymes recognize a sequence specific sites (recognition site) in DNA. Typically the recognition site varies from enzyme to enzyme and may also vary in length. Isoschizomers are enzymes that share the same recognition site. Restriction enzymes may cleave close to or within their recognition site or outside of the recognition site. Often the recognition site is symmetric because the enzyme binds the double stranded DNA as homodimers. Recognition sequences may be continuous or may be discontinuous, for example, two half sites separated by a variable region. Cleavage can generate blunt ends or short single stranded overhangs.

In a preferred aspect one or more restriction enzymes with degenerate recognitions sites are used. Such enzymes include, for example, BstN I, Ban I, BsrFI, BstE II, AlwN I, Rsr II, Ban II and Sty I. For additional enzymes and their recognition sites see the New England BioLabs catalogue.

A number of methods disclosed herein require the use of one or more “restriction enzymes or endonucleases” to fragment the nucleic acid sample. In general, a restriction enzyme recognizes a specific nucleotide sequence of four to eight nucleotides and cuts the DNA at a site within or a specific distance from the recognition sequence. For example, the restriction enzyme EcoRI recognizes the sequence GAATTC and will cut a DNA molecule between the G and the first A. The length of the recognition sequence is roughly proportional to the frequency of occurrence of the site in the genome. A simplistic theoretical estimate is that a six base pair recognition sequence will occur once in every 4096 (4⁶) base pairs while a four base pair recognition sequence will occur once every 256 (4⁴) base pairs. If an enzyme with a variable position in the recognition site is used this changes the frequency of occurrence. For example, Sty1 has recognition site CCWWGG where W can be A or T so a theoretical estimate for the frequency of occurrence of the site is once every 1024 (4⁴×2²) bases. In silico digestions of sequences from the Human Genome Project show that the actual occurrences may be more or less frequent, depending on the sequence of the restriction site. Because the restriction sites are rare, the appearance of shorter restriction fragments, for example those less than 1000 base pairs, is much less frequent than the appearance of longer fragments. Many different restriction enzymes are known and appropriate restriction enzymes can be selected for a desired result. For a comprehensive list of many commercially available restriction enzymes, their recognition sites and reaction conditions see, New England BioLabs Catalog which is herein incorporated by reference in its entirety for all purposes.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

The term “wafer” as used herein refers to a substrate having surface to which a plurality of arrays are bound. In a preferred embodiment, the arrays are synthesized on the surface of the substrate to create multiple arrays that are physically separate. In one preferred embodiment of a wafer, the arrays are physically separated by a distance of at least about 0.1, 0.25, 0.5, 1 or 1.5 millimeters. The arrays that are on the wafer may be identical, each one may be different, or there may be some combination thereof. Particularly preferred wafers are about 8″×8″ and are made using the photolithographic process.

### Methylation Analysis

Mammalian methylation patterns are complex and change during development, see van Steensel and Henikoff BioTechniques 35: 346-357 (2003). Methylation in promoter regions is generally accompanied by gene silencing and loss of methylation or loss of the proteins that bind to the methylated CpG can lead to diseases in humans, for example, Immunodeficiency Craniofacial Syndrome and Rett Syndrome, Bestor (2000) Hum. Mol. Genet. 9:2395-2402. DNA methylation may be gene-specific and occurs genome-wide.

Methods for detecting methylation status have been described in, for example U.S. Pat. Nos. 6,214,556, 5,786,146, 6,017,704, 6,265,171, 6,200,756, 6,251,594, 5,912,147, 6,331,393, 6,605,432, and 6,300,071 and US Patent Application publication Nos. 20030148327, 20030148326, 20030143606, 20030082609 and 20050009059, each of which are incorporated herein by reference. Other array based methods of methylation analysis are disclosed in U.S. patent application Ser. No. 11/058,566.

Many methods used for studying DNA methylation employ methylation sensitive enzymes or bisulfite conversion to detect methylated cytosines at CG dinucleotides. A number of techniques employ 5 methyl cytosine binding proteins or anti-5-methyl cytosine antibodies to biochemically pull down or detect methylated cytosines by immunofluorescence. DNA which has been enriched for methylated regions using affinity based pull down methods can be further characterized using DNA microarrays. Amplification of the enriched DNA and hybridization of the amplified material onto an array can be used to identify which regions of the genome were enriched by the pull-down procedure. In many cases, the majority of cytosines occurring within CG dinucleotide sequences can be methylated (for example, some estimates put this at 70 to 90%). An efficient pull down would thus not result in substantial complexity reduction. For large genomes such as human it may be useful to reduce the complexity of the sample prior to hybridization analysis to minimize cross hybridization and reduce incubation times required for the hybridization to reach equilibrium. Methods are disclosed herein for complexity reduction of affinity enriched methylated DNA. The methods allow for a genome-wide survey of DNA methylation. The disclosed methods may be used to reduce the complexity of samples enriched for other features as well.

For a review of some methylation detection methods, see, Oakeley, E. J., Pharmacology & Therapeutics 84:389-400 (1999). Available methods include, but are not limited to: reverse-phase HPLC, thin-layer chromatography, SssI methyltransferases with incorporation of labeled methyl groups, the chloracetaldehyde reaction, differentially sensitive restriction enzymes, hydrazine or permanganate treatment (m5C is cleaved by permanganate treatment but not by hydrazine treatment), sodium bisulfite, combined bisulphate-restriction analysis, and methylation sensitive single nucleotide primer extension.

In one aspect the methods of the invention relate to methods that result in enrichment and amplification of methylated sequences from a genomic sample. The amplified sample has sequence complexity that is reduced from the starting genome, for example, the complexity may be less than 50%, 25% or 10% of the starting sample and the reduced complexity sample may then be interrogated to determine the methylation status of a plurality of positions in the starting sample. In many aspects interrogation is by hybridization to a high density array of oligonucleotide probes. The methylation state of a plurality of sequences can be determined using the methods. Preferably more than 1,000, 5,000, 10,000, or more than 100,000 different cytosines are analyzed for methylation in parallel. The methods may be used to identify biomarkers of epigenetic regulation based on methylation of surrounding CpGs.

In general methods for obtaining a reduced complexity genomic sample that is enriched for methylated DNA is disclosed. In a further aspect the enriched sample is analyzed for methylation, preferably by hybridization to a nucleic acid array. The methods provide for genome-wide analysis of methylation.

In many embodiments selective adaptor ligation (SAL) amplification is used to reduce the complexity of a genomic sample by amplifying a subset of known fragments. SAL is a method that has previously been used for complexity reduction of samples for genotyping analysis. Briefly, the method includes digestion of the genomic DNA with a restriction enzyme that has degeneracy in its recognition sequence, at least one degenerate position occurring in the overhang generated by cleavage. The complexity of the digested DNA can be reduced by selectively ligating to the fragments adaptors that only ligate to a subset of the generated ends. The subset is modulate by choosing one or more adaptors that can base pair with only one, tow or three bases at the degenerate position of the overhang created by digestion.

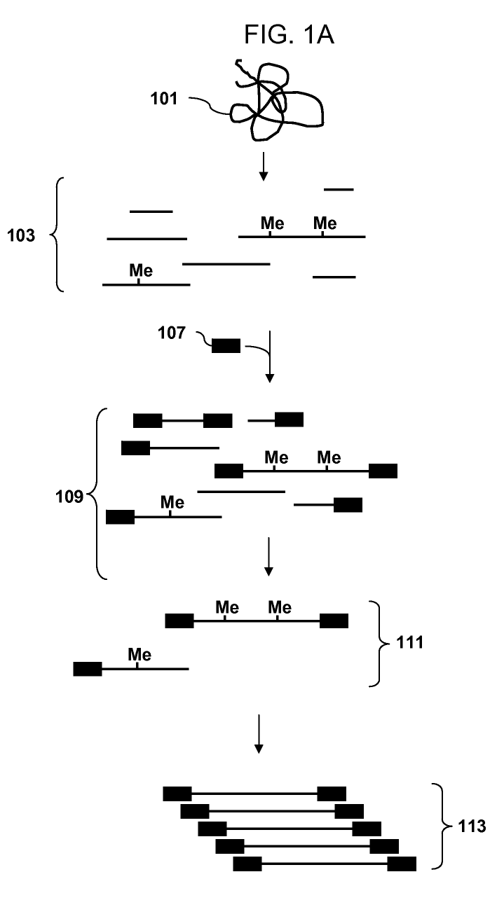

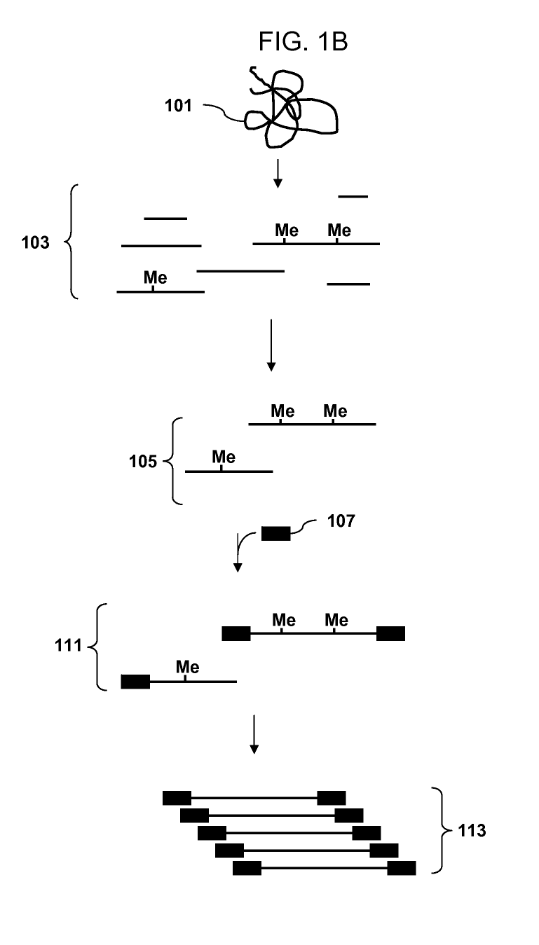

Methylated fragments in the sample are separated from non-methylated fragments and the methylated, amplified fragments are analyzed by hybridization to identify methylated fragments. Schematics of two similar embodiments are shown in FIGS. 1a and 1b. The genomic DNA 101 is fragmented using a restriction enzyme with a degenerate recognition site to obtain restriction fragments 103. The fragments are a mixture of methylated fragments (labeled with “Me”) and unmethylated fragments. In one embodiment, shown in FIG. 1a, selective adaptor 107 is first ligated to the restriction fragments to form adaptor-ligated fragments 109. The adaptor ligated fragments are then subjected to affinity purification to enrich for methylated fragments 111. In an alternative embodiment, shown in FIG. 1b, the restriction fragments 103 are first subjected to affinity purification to enrich for methylated fragments 105 and then the methylated fragments are ligated to the adaptor 107 to form adaptor ligated fragments 111. The methylated, adaptor ligated fragments 111 are then amplified by PCR using a primer to adaptor 107 to obtain a sample that is enriched for a subset of fragments 113. The fragments that are amplified are those that were methylated in the starting sample and ligated to adaptor 107 on both ends. The amplified fragments 113 no longer contain methyl cytosine, but the fragments can be detected, for example, by hybridization, and the presence of a fragment indicates that the fragment was methylated in the starting sample.

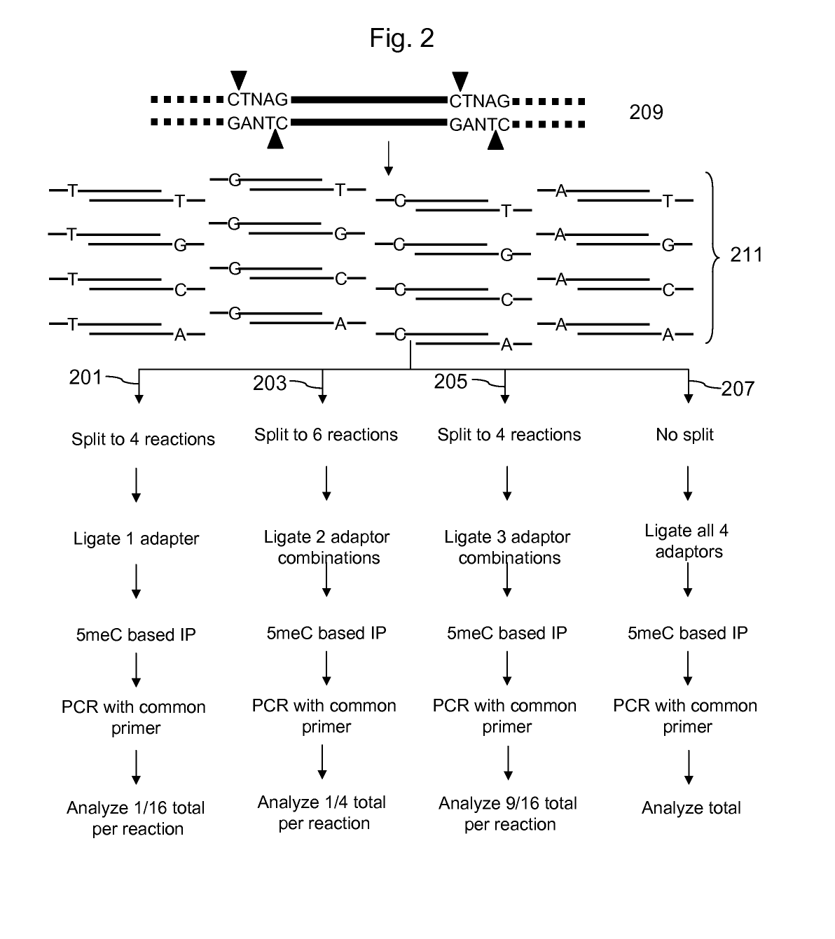

SAL is described, for example, in U.S. patent application Ser. No. 11/381,125, PGPub No. 20060292597 A1. Briefly, the SAL method uses digestion of the DNA with a restriction enzyme that has degeneracy in its recognition sequence that results in degeneracy in at least one position of the single-stranded overhang generated by digestion. For example, the recognition site for DdeI is CTNAG where N can be A, G, C or T. Digestion with DdeI results in a single stranded overhang of TNA or 4 different possible overhangs, TTA, TGA, TCA and TAA. Each DdeI restriction fragment will have two DdeI generated overhangs (one at either end of the fragment) and with the 4 possible overhangs there are 16 end combinations, but only 10 different possible combinations of ends that can result (4+3+2+1=10). See FIG. 2.

The complexity of the DdeI digested DNA can be reduced in a predictable and reproducible way by ligating adaptors to only a subset of the ends and then amplifying only those fragments that have adaptors ligated to both ends. In FIG. 2, four different methods (201, 203, 205 and 207) for generating samples of different complexity are shown. In each of the methods the genomic DNA 209 is fragmented with a restriction enzyme that has a degenerate recognition site to produce a population of fragments with different ends as shown by population 211. In the first method, 201, the fragmented sample is divided into 4 separate tubes and a single adaptor is ligated to the fragments in each tube. If a single adaptor is added only those fragments that have that adaptor ligated to both ends will be amplified [201]. Assuming for simplification purposes that A, G, C and T occur at N at approximately the same frequency a single adaptor will amplify about 1/16^{th} of the amplifiable fragments in any given tube and ¼ of the fragments being amplified across the four tubes. A combination of two adaptors [203] will amplify approximately ¼ of the amplifiable fragments. For example, if an adaptor with an A at the N position and an adaptor with a C at the N position are combined, the fragments that have T at both ends, G at both ends or T at one end and G at the other end will all be targets for amplification. There are 6 possible combinations of two adaptors. Each different combination results in amplification of a different fraction of the genome. In some aspects a single reaction may be analyzed or one or more fractions may be combined for analysis. Fragments are “amplifiable” if they can be amplified by the selected amplification method, for example, when PCR is used, larger fragments (greater than about 2 kb) and smaller fragments (less than about 200 base pairs) may not be amplified efficiently. With a combination of 3 adaptors in a reaction (205) about 9/16 of the amplifiable fragments may be amplified in each reaction. In another aspect (207) all four possible adaptors may be ligated in a single reaction. In this aspect all fragments in the amplifiable size range are available for amplification.

In one aspect genomic DNA is fragmented with an enzyme that has a degenerate recognition site and an affinity pull-down is used to enrich for fragments that contain methyl cytosine. The material that is pulled down is then ligated to a subset of the complementary adaptors and those fragments that have adaptors ligated to both ends are amplified. The amplified product can then be analyzed to identify fragments that are present. Only those fragments that contained methyl cytosine should be detected above background.

In another embodiment the digested fragments may be ligated to the adaptor or adaptors first and then subjected to affinity separation of the fragments that contain 5 methyl cytosine.

In another aspect the complexity reduction is accomplished by using overhang specific primers. The genomic DNA is digested with an enzyme with a degenerate recognition site, adaptors complementary to all overhangs are ligated to the fragments, methyl cytosine containing fragments are isolated by affinity purification and a subset of the affinity purified fragments are amplified by PCR using a primer or primers that are complementary to a subpopulation of fragments. In this aspect the primers vary only in the base that pairs with the degenerate position in the restriction site.

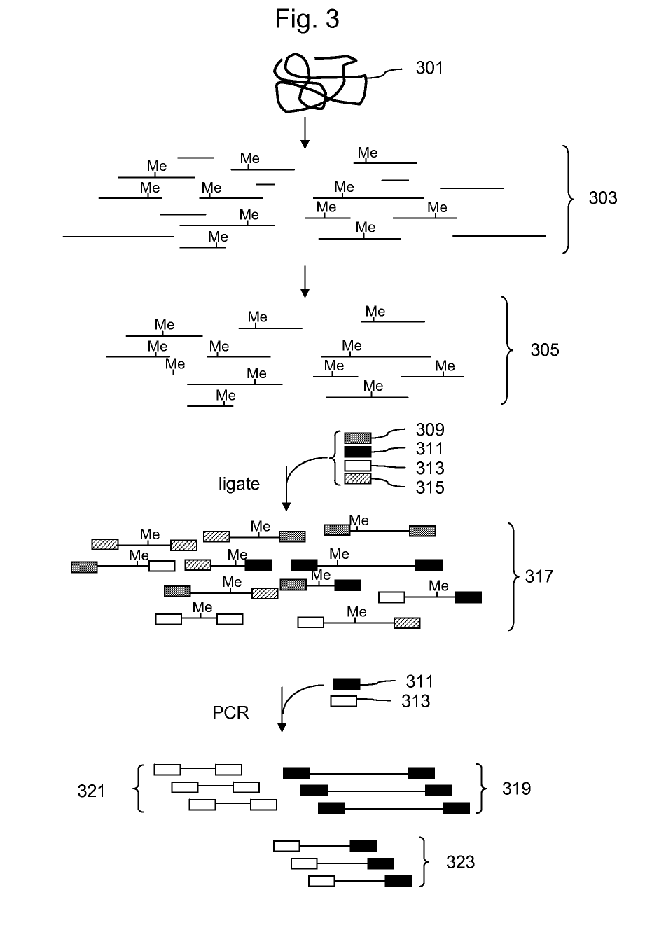

In another embodiment, shown in FIG. 3, the adaptor sequences vary in both the priming sequence and in the overhang sequence so that a different priming sequence is attached to each different type of overhang. The genomic DNA 301 is fragmented to produce a mixture of fragments 303 using a restriction enzyme (RE) that has a degenerate recognition site. Affinity selection for methylated fragments is used to generate enriched sample 305. The degenerate recognition sequence results in four different overhangs that vary by a single base in the restriction fragments. Each different overhang is targeted by a different adaptor (309, 311, 313 and 315) each with a different overhang and a different priming sequence. All of the adaptors may be used in the ligation, as shown, and adaptor-ligated fragments with each of the adaptors will be generated to form population 317. If the amplification is performed using only two primers that are complementary to adaptors 311 and 313 then only those fragments with those adaptors ligated will be amplified (319, 321 and 323). The affinity selection of methyl cytosine containing fragments can take place before adaptor ligation as shown in FIG. 3 or after adaptor ligation but before amplification. The amplification product may be analyzed to detect the presence of selected fragments.

In many aspects the amplification product is labeled by a detectable label before hybridization to an array. The amplification product may be fragmented and the fragments may be labeled, for example, by end labeling using TdT and incorporating a biotin labeled nucleotide. The labeled fragments may be hybridized to an array of probes and the pattern of hybridization can be analyzed to determine methylation.

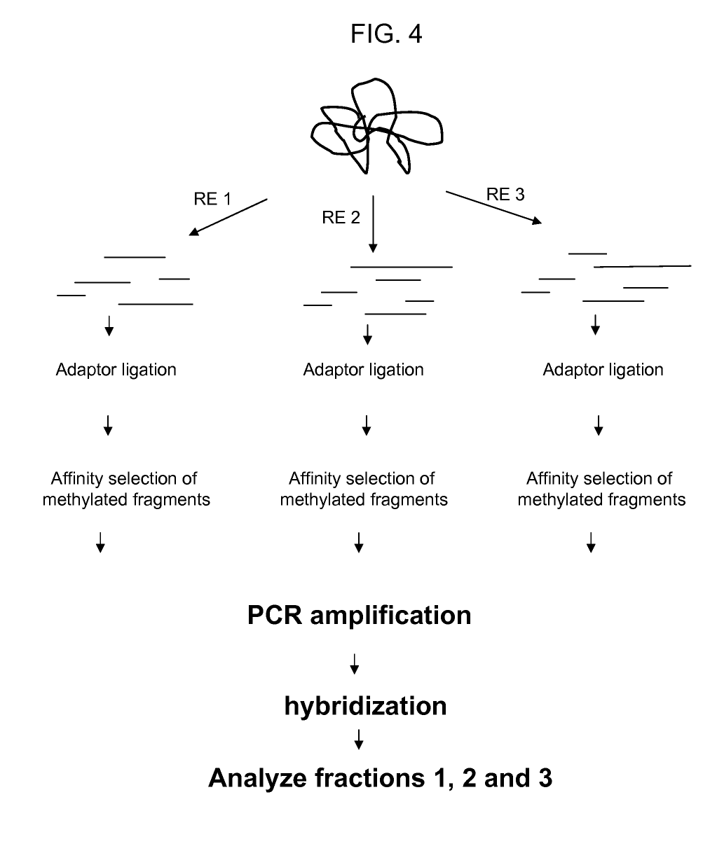

In another aspect, shown in FIG. 4, separate aliquots of genomic DNA are digested with different enzymes. Adaptors are ligated to the fragments generated in each digest. An affinity based pull down of methylated fragments is performed and size-selective PCR is performed on the isolated DNA. The size selective PCR is a complexity reducing step. For each different enzyme used to digest the genomic DNA a distinct population of fragments are amplified by the size-selective PCR. The different fractions can be hybridized to the same or separate arrays. The complexity can be modulated by the choice of enzymes. In a preferred aspect digests from two, three or more enzymes are combined. The reactions may be combined after amplification or after digestion but prior to amplification.

Many of the embodiments may include one or more steps of computer implemented in silico digestion. In silico digestion typically involves analysis of the sequence of a genome or genomic region to locate the recognition sites for a selected restriction enzyme or combination of enzymes and predicting the sizes and sequences of the fragments that will result from digestion of a sample with the selected enzyme or enzyme combination. The output of the in silico digestion may be, for example, an electronic file reporting the sequence of predicted fragments. In one aspect a computer is used to identify the fragments that result when a genome is digested with an enzyme that has a degenerate restriction site. The different combinations of ends may also be modeled by the computer to predict which fragments would be amplified in a given adaptor-ligation and amplification scheme as described above. A computer may also be used to identify fragments that are amenable to amplification by the PCR conditions. In many embodiments the PCR conditions preferentially amplify fragments of a limited size range, for example, 100, 200 or 400 to 800, 1,000 or 2,000 base pairs. Fragments that are within the expected size range and contain a site for a methylation sensitive enzyme are identified and an array may be designed with probes complementary to a plurality of the fragments that are identified.

The approaches generally target degeneracy in the enzyme recognition site but not the sequence diversity of the fragments themselves. Reducing the complexity of the sample prior to hybridization improves signal to noise but also reduces the number of sequences that can be interrogated. Current methods for genotyping are able to genotype large numbers of SNPs simultaneously and thus require a minimal level of complexity to provide the large numbers of targets for genotyping. Reducing the complexity also reduces the amount of information that can be interrogated.

In one aspect the enzyme Nsp I is used. The recognition site is 5′ RCATGVY 3′. All possible recognition sequences with this consensus sequence may be cut, not just those that are palindromic. Sequences include ACATGC, ACATGT, GCATGC, and GCATGT. The enzyme cuts more frequently than a 6 cutter but less frequently than a 4 cutter, providing a complexity reduction that is more than a 4 cutter and less than a 6 cutter, providing The 3′ overhang has some constant and some variable positions.

The recognition site for the enzyme Dde I is GTNAG, where N can be A, C, G or T. The possible combinations of sequences recognized by Dde I are CTAAG, CTCAG, CTGAG and CTTAG. Digestion with Dde I generates 3 base pair 5′ overhangs with the sequence 5′ TNA 3′. The possible resulting overhangs are TAA, TCA, TGA and TTA. Restriction fragments resulting from Dde I digestion can have 16 possible combinations of Dde I sites (any one of the 4 possible sites on either of the two ends).

In one aspect genomic DNA is fragmented with a restriction enzyme that contains at least one degenerate position in the restriction site and an adaptor that has at least one degenerate position, corresponding to a degenerate position in the restriction site is ligated to the fragments. A subset of the fragments is amplified using a primer that is not degenerate at the position corresponding to the degenerate position in the restriction site or only partially degenerate at that position, i.e. including less than all possible combinations of sequence at the degenerate position or positions.

Fragmentation with an enzyme that recognizes a sequence that contains at least one degenerate position results in more frequent cutting of the DNA. For example, an enzyme with a 5 base pair recognition sequence will cleave on average once every 45 bases or on average every 1024 bases. If one of the 5 bases can be any base the enzyme will cleave on average every 256 bases, similar to using an enzyme with a recognition sequence of 4 bases. The enzyme with a degenerate base in the recognition sequence allows for an additional level of selection, over a 4 base cutter, because the overhang has a degenerate position that can be used selectively for adaptor ligation or for hybridization of a primer in an amplification reaction.

Selective adaptor ligation may be used to control the complexity by varying the adaptors that are included in the ligation. For example, when DdeI is used the enzyme has a recognition site GTNAG where N can be A, G, C or T. Restriction fragments resulting from DdeI will be flanked on both ends by a DdeI overhang. The two overhangs may have any of the ten possible combinations of two of the four possible bases at the N₁ and N₂ positions. The ten possible combinations for (N₁,N₂) are (A,A), (A, G), (A,C), (A,T), (G,G), (G, C), (G, T), (C, C), (C, T) and (T, T). If adaptors with a T or C at the position complementary to the N are included during the ligation they will ligate to restriction sites that had either an A or a G at the N (A, A), (A, G) and (G, G). For the fragment to be amplified it should have the adaptor sequence ligated to both ends so if a single adaptor sequence is added, for example, with an overhang of ATT only 10% of the fragments are targets for amplification (only those fragments that have the 5′-TAA-3′ overhang on both ends). If two adaptor overhangs are used, for example 3′-ATT-5′ and 3′-ACT-5′ approximately 30% of the fragments will be targets for amplification (those fragments that have either 5′-TAA-3′ on both ends, those fragments that have 5′-TGA-3′ on both ends or fragments that have 5′-TAA-3′ on one end and 5′-TGA-3′ on the other end. Different combinations of adaptors may be used to amplify different collections of fragments and to interrogate the polymorphisms in those different collections. The population of polymorphisms that are present on fragments that have A or G at the N position on both ends is different from the population of polymorphisms that are present on fragments that have C or T at the N position on both ends.

The sequence of the human genome and many other organisms is known and publicly available so computer simulations of restriction digests can be used to predict the fragments that will be amplified and to identify the polymorphisms that will be in the amplified fraction, given a selected combination of adaptor sequences.

The use of selective ligation of adaptors allows for many different possible combinations that can be used to fine tune the complexity of the resulting amplification product. For example, an adaptor that is complementary to only one of the possible sequences left by digestion may be used. If there is a single degenerate position that can be any of the 4 possible bases and the adaptor is complementary to just one of the 4 possible overhangs then about 25% of the restriction sites will have the adaptor ligated to them and only those fragments that have the adaptor ligated to both ends will amplify. This adds an additional layer of complexity reduction to the size based complexity reduction of the WGSA.

In another embodiment an enzyme with more than one degenerate base in the recognition sequence may be used, for example, BsaJ1 may be used. The recognition site for BsaJ1 is C^{V}CNNGG. The 5′ overhang after digestion is CNNG where both N's can be either A, C, G or T. There are 16 different overhangs possible. Adaptors can be selected to target different populations of fragments for amplification and analysis. Using in silico digestion methods the fragments that will be amplified when a particular adaptor or combination of adaptors is used can be predicted based on the sequence. Those fragments that will be amplified are possible targets and SNPs that are within those fragments are targets for genotyping.

In some embodiments complexity reduction by isolation of methylated sequences and selective adaptor ligation is combined with AFLP (Keygene, NV). AFLP is described in U.S. Pat. Nos. 6,045,994 and 6,300,071. In the AFLP method one of the primers used to amplify adaptor ligated affinity selected fragments is complementary in part to at least one base within the restriction fragment and outside of the recognition site for the restriction enzyme.

In some embodiments methylation analysis includes a step where the genomic DNA sample is subjected to treatment with bisulfite. Unmethylated cytosine is converted to uracil through a three-step process during sodium bisulfite modification. The steps are sulphonation to convert cytosine to cytosine sulphonate, deamination to convert cytosine sulphonate to uracil sulphonate and alkali desulphonation to convert uracil sulphonate to uracil. Conversion on methylated cytosine is much slower and is not observed at significant levels in a 4-16 hour reaction. See Clark et al., Nucleic Acids Res., 22(15):2990-7 (1994). If the cytosine is methylated it will remain a cytosine. If the cytosine is unmethylated it will be converted to uracil. When the modified strand is copied, through, for example, extension of a locus specific primer, a random or degenerate primer or a primer to an adaptor, a G will be incorporated in the interrogation position (opposite the C being interrogated) if the C was methylated and an A will be incorporated in the interrogation position if the C was unmethylated. When the double stranded extension product is amplified those Cs that were converted to U's and resulted in incorporation of A in the extended primer will be replaced by Ts during amplification. Those Cs that were not modified and resulted in the incorporation of G will remain as C.

Kits for DNA bisulfite modification are commercially available from, for example, Human Genetic Signatures' Methyleasy and Chemicon's CpGenome Modification Kit. See also, WO04096825A1, which describes bisulfite modification methods and Olek et al. Nuc. Acids Res. 24:5064-6 (1994), which discloses methods of performing bisulfite treatment and subsequent amplification on material embedded in agarose beads. In one aspect a catalyst such as diethylenetriamine may be used in conjunction with bisulfite treatment, see Komiyama and Oshima, Tetrahedron Letters 35:8185-8188 (1994). Diethylenetriamine has been shown to catalyze bisulfite ion-induced deamination of 2′-deoxycytidine to 2′-deoxyuridine at pH 5 efficiently. Other catalysts include ammonia, ethylene-diamine, 3,3′-diaminodipropylamine, and spermine. In some aspects deamination is performed using sodium bisulfite solutions of 3-5 M with an incubation period of 12-16 hours at about 50° C. A faster procedure has also been reported using 9-10 M bisulfite pH 5.4 for about 10 minutes at 90° C., see Hayatsu et al., Proc. Jpn. Acad. Ser. B 80:189-194 (2004).

Bisulfite treatment allows the methylation status of cytosines to be detected by a variety of methods. For example, any method that may be used to detect a SNP may be used, for examples, see Syvanen, Nature Rev. Gen. 2:930-942 (2001). Methods such as single base extension (SBE) may be used or hybridization of sequence specific probes similar to allele specific hybridization methods. In another aspect the Molecular Inversion Probe (MIP) assay may be used.

In a preferred aspect, molecular inversion probes, described in Hardenbol et al., Genome Res. 15:269-275 (2005) and in U.S. Pat. No. 6,858,412, may be used to determine methylation status after methylation dependent modification. A MIP may be designed for each cytosine to be interrogated. In a preferred aspect the MIP includes a locus specific region that hybridizes upstream and one that hybridizes downstream of an interrogation site and can be extended through the interrogation site, incorporating a base that is complementary to the interrogation position. The interrogation position may be the cytosine of interest after bisulfite modification and amplification of the region and the detection can be similar to detection of a polymorphism. Separate reactions may be performed for each NTP so extension only takes place in the reaction containing the base corresponding to the interrogation base or the different products may be differentially labeled.

In a preferred aspect the products are analyzed by hybridization to an array. In one exemplary embodiment an array is designed to detect the products of bisulfite modification using the same principles as the commercially available Affymetrix 10K Mapping Array. The 10K array has probe sets for each of more than 11,000 different human SNPs. Each probe set has a first plurality of probes that are perfectly complementary to a first allele of the SNP and a second plurality of probes that are perfectly complementary to the second allele of the SNP. If the first allele is present signal is detected by the first plurality of probes and if the second allele is present signal is detected by the second plurality of probes. Heterozygotes result in signal detection by both. The probe sets may include control probes, for example, mismatch probes, probes that shift the interrogation position relative to the central position of the probe may be included, for example, the SNP position may be at the central position or it may be shifted 1 or more positions 5′ or 3′ of the center of the probe. Analogous probe sets could be designed for suspected sites of methylation, treating the position as though it were a SNP with alleles C/G or T/A. Both strands may be analyzed. Exemplary probes and arrays are described in US PGPub No. 20040146890 and U.S. Pat. Nos. 5,733,729, 6,300,063, 6,586,186, and 6,361,947. The bisulfite treatment can modify any unmethylated C in the fragments, including C's in primer binding sites and C's that are in regions surrounding an interrogation positions. In preferred embodiments the adaptors are designed to take this into account, for example, the adaptor may be designed so that there are no C's in the primer binding site, the primer may also be synthesized with modified bases that are resistant to bisulfite modification so that the sequence of the primer binding site is not changed by the treatment, for example, C's could be methylated, or the primer can be designed assuming that the C's in the adaptor will be changed to U's.

Resequencing arrays which allow detection of novel SNPs from a sequence may also be used to detect the products of the bisulfite treatment. Resequencing arrays and resequencing methods are described, for example, in Cutler et al. Genome Res. 2001 November; 11(11): 1913-25 and in US patent publication No. 20030124539, both of which are incorporated herein by reference in their entirety. In general resequencing arrays detect all possible single nucleotide variations in a reference sequence. Probes are included that are perfectly complementary to the reference sequence and interrogate a plurality of positions in the sequence individually for variation in the reference sequence. Probes that are perfectly complementary to the variant sequence are included for each possible variation. An array may be tiled to detect all possible single nucleotide variations in one or more reference sequences. To detect the products of bisulfite treatment, instead of designing probes to all possible single nucleotide variants, the probes may be designed to detect possible variations at cytosines, depending on methylation. The reference sequence or sequences interrogated by the array may be, for example, one or more entire chromosomes, one or more entire genomes, one or more mitochondrial genomes, or selected regions of interest from within one or more genomes. In one embodiment a resequencing array is tiled with regions that are known or suspected to be methylated. In some embodiments CpG sites may be close together so that the probes of the array may be complementary to overlapping CpG sites. For example if the probe is a 25 mer and the interrogation position at position 13 is complementary to a first cytosine position there may be a second CpG that is within the 12 base pairs upstream or the 12 base pairs downstream of the first cytosine. The second cytosine may or may not be methylated. Probes can be designed to detect both possibilities, i.e. both methylated (both C), both unmethylated (both T), one methylated (C) and the other unmethylated (T). Probes that are perfectly complementary to each possible outcome may be designed.

In some aspects of the invention amplified methylated target is enriched relative to unmethylated target. In one aspect, antibodies to 5-meC are used to isolate adaptor-ligated fragments that contain 5-meC. Alternatively the nucleic acid may be incubated with proteins that specifically bind 5-meC and then antibodies to those proteins may be used to isolate methylated fragments. Antibodies to 5-meC are available, for example, from Abcam (Cambridge, UK), for example, ab1884 and ab10805 (5-Methyl Cytidine antibody [clone 33D3] and from Aviva Systems Biology, for example, AMM99021. This is a mouse IgG1 isotype monoclonal antibody, For methods of using 5-methyl cytidine antibodies see, for example, Pfarr et al., Biotechniques 38:527-8, 530 (2005), Hernandex-Blazquez et al., Gut 47:689-93 (2000), Habib et al., Exp Cell Res 249:46-53 (1999), Fraga et al., Cancer Res. 64(16):5527-34 (2004) and Reynaud, et al., Cancer Lett 61:255-62 (1992), each of which is incorporated herein by reference in its entirety.

Affinity isolated fragments are amplified by PCR using a primer complementary to the adaptor and the amplified fragments may be hybridized to an array of probes. In a preferred aspect the probes of the array are complementary to one or more regions of the genome. Regions of the array that show hybridization above background are indicative of areas of the genome that are methylated. In a preferred embodiment the array comprises probes to CpG rich regions of the genome, intragenic regions, or regions known or predicted to be regulatory regions. In a preferred aspect the array may be a CpG island array as disclosed in U.S. patent application Ser. No. 11/695,599. The reduced complexity, methylated fragment enriched sample may also be analyzed using a promoter array or a tiling array. Promoter and CpG island arrays and methods of using these arrays and preparing samples for hybridization to these arrays are disclosed in “Promoter and CpG Island Microarrays (Nuts & Bolts series) Eds. Takahashi and Winegarden, DNA press (2005). In another embodiment the immunoprecipitated fragments are treated with bisulfite so that precise locations of methylated cytosines may be identified. The sample may be analyzed by hybridization to an array of sequence specific probes as described above.

In one aspect of the invention methyl binding proteins, such as MeCP2 and SAP18/30 (Sin3 associated Polypeptides 18/30), are mixed with the genomic DNA sample and used to enrich for methylated sequences. Antibodies to methyl CpG binding domain proteins (MBDs), for example, MBD2 and MBD3 may be used to isolate DNA containing methylation. MBD1 and MBD4 are also methyl binding proteins. Antibodies against 5-meC-binding proteins are available, for example, antibodies to MeCP2 (IMG-297) are available from Imgenex Corp. (San Diego, Calif.). In another aspect antibodies that recognize 5-meC may be used to enrich for methylated sequences. The DNA is preferably denatured prior to antibody binding. Methyl-CpG-binding proteins and methods of analysis are disclosed, for example, in Ballestar and Wolffe, Eur. J. Biochem. 268:1-6 (2001), Fournier et al., EMBO J. 21:6560 (2002) and Ballestar et al., EMBO J. 22:6335-6345 (2003).

Methods for separation of methylated from unmethylated nucleic acids have been described, see, for example, US patent publication nos. 20010046669, 20030157546, and 20030180775 which are each incorporated herein by reference in their entireties. Methods for detection and analysis of DNA methylation are also disclosed in Brena et al., J. Mol. Med. 2006 Jan 17:1-13 [Epub ahead of print].

A number of methyl-dependent restriction enzymes are known to those of skill in the art and are available commercially from, for example, New England Biolabs. Examples of methyl-dependent restriction enzymes include, McrBC, McrA, MrrA, and DpnI. McrBC is an endonuclease which cleaves DNA containing methylcytosine, (e.g. 5-methylcytosine or 5-hydroxymethylcytosine or N4-methylcytosine, reviewed in Raleigh, E. A. (1992) Mol. Microbiol. 6, 1079-1086) on one or both strands. McrBC will not act upon unmethylated DNA (Sutherland, E. et al. (1992) J. Mol. Biol. 225, 327-334). The recognition site for McrBC is 5′ . . . Pu^{m}C(N₄₀₋₃₀₀₀) Pu^{m}C . . . 3′. Sites on the DNA recognized by McrBC consist of two half-sites of the form (G/A)^{m}C. These half-sites can be separated by up to 3 kb, but the optimal separation is 55-103 base pairs (Stewart, F. J. and Raleigh E. A. (1998) Biol. Chem. 379, 611-616 and Panne, D. et al. (1999) J. Mol. Biol. 290, 49-60). McrBC requires GTP for cleavage, but in the presence of a non-hydrolyzable analog of GTP, the enzyme will bind to methylated DNA specifically, without cleavage (Stewart, F. J. et al. (2000) J. Mol. Biol. 298, 611-622). Recombinant McrBC is available from, for example, New England Biolabs. McrBC may be used to determine the methylation state of CpG dinucleotides. McrBC will act upon a pair of Pu^{m}CG sequence elements, but will not recognize Hpa II/Msp I sites (CCGG) in which the internal cytosine is methylated. The very short half-site consensus sequence (Pu^{m}C) allows a large proportion of the methylcytosines present to be detected.

In one embodiment reaction conditions for digestion with McrBC are 50 mM NaCl, 10 mM Tris-HCl, 10 mM MgCl₂, 1 mM dithiothreitol (pH 7.9 at 25° C.) with 100 μg/ml BSA and 1 mM GTP. Incubate at 37° C. Conditions may be varied. NEB defines one unit as the amount of enzyme required to cleave 1 μg of a plasmid containing a single McrBC site in 1 hour at 37° C. in a total reaction volume of 50 μl. A 5 to 10-fold excess of enzyme may be used for cleavage of genomic DNA. The enzyme may be heat inactivated by heating to 65° C. for 20 minutes. McrBC makes one cut between each pair of half-sites, cutting close to one half-site or the other, but cleavage positions are distributed over several base pairs approximately 30 base pairs from the methylated base. See also, Bird, A. P. (1986) Nature 321, 209-213 and Gowher, H. et al. (2000) EMBO J. 19, 6918-6923.

Studies on or utilizing McrBC have been reported in the literature, for example, Gast et al. Biol Chem. 378(9):975-82, (1997), Pieper et al., Rabinowicz, Methods Mol Biol. 236:21-36 (2003), Badal et al. J. Virol. 77(11):6227-34 (2003) and Chotai and Payne, J Med Genet. 35(6):472-5 (1998). See also, Lyko, F. et al. Nat. Genet., 23, 363-366 (2000) which used McrBC as a tool for enrichment of undermethylated DNA in drosophila.

In one aspect the disclosed methods are used to obtain a methylation signature or profile of a tumor or tissue. Methylation is of particular interest in the diagnosis, treatment and outcome prediction for cancer, see Jones and Baylin, Nat. Rev. Genet. 3:415-428 (2002) and Bird, Genes Dev. 16:6-21 (2002). Patterns of methylation may be associated with specific tumors. Samples from a specific type of tumor may be isolated and analyzed using the methods disclosed to obtain a methylation pattern characteristic of a tumor type or the stage of a tumor. In one embodiment a sample from an individual or from a tumor may be compared to the methylation pattern of a tumor of known type or stage to determine if the unknown sample is similar to one or more of the known tumor types in methylation pattern. Patterns obtained according to the methods may be used to diagnose disease, stage disease, monitor treatment, predict treatment outcome, and monitor disease progression. In many embodiments analysis is performed by a direct comparison of a hybridization pattern without correlation of the pattern to the presence or absence of any specific sequence. Differences or similarities between a pattern obtained from an unknown sample that is being analyzed and patterns obtained from known samples can be used to determine if the unknown is likely to match the known sample in methylation pattern.

In one embodiment blood samples are analyzed to detect changes in the methylation pattern of tumor cells that are sloughed-off into the blood stream. Patterns of aberrant methylation or demethylation that are characteristic of a tumor type may be identified by analysis of a blood sample. Aberrant methylation patterns may be correlated with cancer, imprinting defects and aging. In one exemplary embodiment the sample is fragmented with a first restriction enzyme and the fragments are ligated to adaptors. The adaptor-ligated fragments are then digested with an enzyme that is methylation dependent or methylation sensitive. The adaptor-ligated fragments that are not digested are amplified by PCR using a primer to the adaptor. The products of the PCR amplification are hybridized to an array of probes to generate a hybridization pattern. The hybridization pattern may be compared to a hybridization pattern from another sample that has been similarly treated. Differences between hybridization patterns are indicative of differences in the methylation patterns between the two samples. A data base of hybridization patterns that are characteristic of disease states, normal states, or tissue types may be generated and used to compare hybridization patterns of unknown samples to identify similar patterns. See, for example, U.S. Pat. No. 6,228,575 which discloses methods of sample characterization based on comparison of hybridization pattern. A variety of arrays may be used for this purpose and it is not necessary that the array be specifically designed to detect specific genomic sequences from the organism being analyzed.

In one embodiment enrichment of unmethylated DNA is combined with comparative genomic hybridization (CGH) to analyze tumor cells to identify differences between tumor DNA and normal DNA. See, for example, Kallioniemi et al. Methods 9(1):113-121 (1996). Equal amounts of differentially labeled tumor DNA and normal reference DNA, (one may be labeled with biotin and the other with digoxigenin, for example), may be hybridized to an array of probes, the signal intensities quantified, and signals that are over or underrepresented in tumor versus normal can be quantified. In one embodiment methods of analysis of methylation status may be combined with methods of estimating copy number of one or more regions of a genome. Many cancers are associated with increases in the copy number of one or more regions of the genome. Increased copy number can be detected by hybridization to arrays. The increase of copy number is detected as an increase in the intensity of hybridization. Methods for analysis of copy number using oligonucleotide arrays are disclosed, for example, in U.S. Patent Pub. No. 20040157243 which discloses specific computer methods to perform copy number analysis using, for example, the GeneChip 10K Mapping Array and the GeneChip Mapping 100K and 500K Array sets and the GeneChip Mapping Assay.

Exemplary arrays that may be used in combination with the disclosed methods include the arrays disclosed in U.S. patent application Ser. Nos. 09/916,135 and 10/891,260 and U.S. Patent Pub. No. 20040067493, each of which is incorporated herein by reference.

In one aspect an array is designed to interrogate methylation status of more than 50,000, more than 100,000, more than 500,000, more than 1,000,000, more than 2,500,000 or more than 5,000,000 of these CpG's. In some embodiments the array may also contain probes to interrogate CNG positions which can also be methylated at the cytosine. Interrogation may be, for example, analogous to detecting a polymorphism at the cytosine position, reflecting the change of the cytosine to a uracil by either chemical, for example bisulfite, or enzymatic, for example AID, mechanisms. Particular CpG's may be selected for interrogation based on the positioning of neighboring CpG dinucleotides. When there are more than one CpG in the region that the probe is complementary to, for example, within the 25 bases of the probe, the perfect complementarity of the probe to interrogate the central CpG may be impacted by the methylation status of the second, third or fourth CpG within the probe region. In some aspects the probe set for interrogation of the first CpG (the interrogation CpG) may be designed to take in all possible combinations of sequence variation resulting from variation in the methylation status of the secondary (non-interrogation) CpGs. This would require additional probes for each possible sequence variation. In another aspect CpGs that do not have another CpG within 12, 15, 20 or 30 bases upstream or downstream are selected for interrogation.

In another aspect, the disclosed methods may be used to detect epigenetic changes in cells that are being grown in cell culture. Cell lines that have been grown in cell culture for many generations may develop epigenetic changes that may alter the expression or growth of the cells, potentially making the cells more prone to formation of tumors, for example. The disclosed methods may be used to analyze cells in culture, for example, cell lines derived from embryonic stem cells to identify epigenetic changes that may impact the usefulness of the cultured cells. The methods may be used for quality control for cell culture.

### EXAMPLE 1

A recommended protocol for IP using ab1884 (Abcam). Use 0.5 to 1 μg of fragmented genomic DNA. Dilute fragmented DNA to 100 μl for a final concentration of 0.15% SDS, 1% triton x-100, 150 mM NaCl, 1 mM EDTA pH8.0, 0.5 mM EGTA pH8.0, mM Tris pH8.0, 0.1% BSA, 7 mM NaOH, anti-5mC (up to 30 ug of antibody for saturating conditions), and Prot A/G beads. Rotate overnight at 4° C. Wash 2× with 0.1% SDS, 0.1% DOC, 1% triton, 150 mM NaCl, 1 mM EDTA pH8.0, 0.5 mM, EGTA pH8.0, 10 mM Tris pH8.0. Wash 1× with 0.1% SDS, 0.1% DOC, 1% triton, 500 mM NaCl, 1 mM EDTA pH8.0, 0.5 mM, EGTA pH8.0, 10 mM Tris pH8.0. Wash 1× with 0.25 M LiCl, 0.5% DOC, 0.5% NP-40, 1 mM EDTA pH8.0, 0.5 mM, EGTA pH8.0, 10 mM Tris pH8.0. Wash 2× with 1 mM EDTA pH8.0, 0.5 mM EGTA pH8.0, 10 mM Tris pH8.0. Elute in 1% SDS, 100 mM NaHCO₃. Purify the DNA and use the isolated DNA for PCR. Analyze the amplicons by hybridization to an array.

### EXAMPLE 2

Obtain a genomic DNA sample. Fragment the sample with Dde I in NEBuffer 3 at 37° C. Heat inactivate the enzyme at 65° C. for 20 minutes. Ligate an adaptor to the fragments. The adaptor has a primer binding site and a single stranded overhang that is 3′-ATT-5′ and will ligate efficiently to the fragment overhangs that have a 5′ TAA-3′ overhang generated by cleavage, but not to the fragment overhangs that have TTA, TCA or TGA. Immunoprecipitate fragments that contain 5-meC using an antibody to 5-meC. Clean up the immunoprecipitated fragments and amplify by PCR using a primer complementary to the primer binding site in the adaptor. Do the PCR in the presence of dUTP so that uracil is incorporated into the DNA. Treat the amplified DNA with uracil DNA glycosylase and APE 1 to fragment the PCR amplicons. End label using terminal deoxynucleotidyl transferase and Affymetrix′ biotin-labeled DNA labeling reagent (DLR). Hybridize the labeled fragments to an array, stain with biotinylated SAPE and anti-streptavidin antibody. Scan to detect hybridization pattern and analyze the hybridization pattern to identify methylated genomic regions.

### CONCLUSION

Methods of analyzing DNA to determine the methylation status of a plurality of cytosines in the genome are disclosed. In preferred aspects the methods include steps of fragmentation, circularization and enrichment of circles with either methylated or unmethylated sites, and detection of sequences in the enriched fraction by hybridization to an array of probes.

The above description is illustrative and not restrictive. Many variations of the invention will become apparent to those of skill in the art upon review of this disclosure. The scope of the invention should, therefore, be determined not with reference to the above description, but instead be determined with reference to the appended claims along with their full scope of equivalents.

We claim:

1. A method for identifying a plurality of methylated genomic regions in a genomic DNA sample, said method comprising:
(a) fragmenting the genomic DNA sample with a restriction enzyme, wherein the recognition site for the restriction enzyme comprises at least one degenerate position, to obtain a population of fragments with a plurality of different single-stranded fragment overhangs;
(b) ligating at least one adaptor to the population of fragments to obtain adaptor-ligated fragments wherein said at least one adaptor comprises a single stranded adaptor overhang that is complementary to one of the fragment overhangs in the plurality of different single-stranded fragment overhangs;
(c) performing an affinity selection for methylated fragments to obtain an enriched sample, wherein said enriched sample is enriched for methylated fragments;
(d) amplifying adaptor-ligated fragments in the enriched sample using a primer to the adaptor, to obtain an amplification product enriched for sequences that were methylated in the sample;
(e) labeling the amplification product with a detectable label;
(f) hybridizing the amplification product to an array of nucleic acid probes; and
(f) determining the methylation status of selected cytosines by analyzing the hybridization pattern.

2. The method of claim 1 wherein the plurality of different overhangs consists of a first, a second, a third and a fourth fragment overhang that each has a different base at the base that is complementary to the degenerate position.

3. The method of claim 2 wherein the at least one adaptor is a single adaptor that has a single-stranded adaptor overhang that is completely complementary to the first fragment overhang and not completely complementary to the second, third or fourth fragment overhangs.

4. The method of claim 3 wherein the at least one adaptor is a first and a second adaptor with single-stranded adaptor overhangs wherein the single-stranded adaptor overhang of the first adaptor is complementary to the first fragment overhang and the single-stranded adaptor overhang of the second adaptor is complementary to the second fragment overhang.

5. The method of claim 1 wherein the array comprises at least 100,000 different oligonucleotide probe sequences, wherein each probe sequence is present at a different known or determinable location in the array and wherein the probes are complementary to fragments in a fraction of the genome wherein the fraction is defined by the presence of restriction sites for a single selected restriction enzyme.

6. The method of claim 5 wherein the probes are each attached to a solid support selected from the group consisting of a bead, a plurality of beads, one or more silica chips and one or more glass slides.

7. The method of claim 1 wherein the step of affinity selection comprises immunoprecipitating by a method comprising mixing the sample with an antibody to 5 methyl cytosine.

8. The method of claim 1 wherein said step of affinity selection comprises immunoprecipitating by a method comprising mixing the sample with a first protein that binds 5 methyl cytosine and an antibody to said first protein.

9. The method of claim 1 wherein said step of affinity selection comprises immunoprecipitating by a method comprising mixing the sample with a protein complex that binds 5 methyl cytosine and an antibody that binds the protein complex.

10. The method of claim 1 wherein the restriction enzyme is selected from the group consisting of Sty1, Nsp1, BsaJI and DdeI.

11. A method of generating a hybridization sample from a genomic DNA sample, wherein the hybridization sample is enriched relative to the genomic DNA sample for fragments that were methylated in the genomic DNA sample, said method comprising:
(a) obtaining a genomic DNA sample;
(b) fragmenting the genomic DNA sample with a restriction enzyme that has at least one degenerate position in the enzyme recognition site, wherein the degenerate position is within a single stranded overhang generated by cleavage with the restriction enzyme;
(c) ligating at least one adaptor sequence to the fragments from (b), wherein the adaptor sequence comprises a primer binding domain and a single stranded fragment overhang that is complementary to at least one of the overhangs generated by cleavage with the restriction enzyme;
(d) immunoprecipitating methylated fragments to obtain a sample enriched for methylated fragments;
(e) amplifying the adaptor ligated fragments with a primer complementary to the primer binding domain of the adaptor;
(f) fragmenting the amplified sample from step (e); and
(g) end labeling the fragments from step (f) to obtain a hybridization sample.

12. The method of claim 11 wherein step (d) is performed after steps (b) and (c).

13. The method of claim 11 wherein step (d) is performed before step (c) and after step (b).

14. The method of claim 11 wherein a first adaptor and a second adaptor are ligated to the fragments in step (c) and wherein the first and second adaptors differ in the position of the overhang that is complementary to the degenerate position in the restriction enzyme recognition site.

15. The method of claim 11 wherein dUTP is included in step (e) and the products of step (e) are fragmented by incubation with uracil DNA glycosidase.

16. A method for identifying a plurality of methylated regions in a genomic DNA sample, the method comprising:
fragmenting the genomic DNA sample with a restriction enzyme, wherein the recognition site for the restriction enzyme comprises at least one degenerate position, to obtain restriction fragments, wherein the restriction fragments comprise a plurality of different sequence overhangs;
performing an affinity selection for restriction fragments that contain a methyl cytosine to obtain a second sample that is enriched for restriction fragments that contain methyl cytosine;
ligating adaptors to at least some of the restriction fragments to obtain adaptor-ligated fragments;
amplifying a subset of the adaptor-ligated fragments from the second sample; and, analyzing the amplified sample to detect the presence of a plurality of genomic regions in the second sample, wherein fragments that are present in the second sample are identified as fragments that were methylated in the genomic DNA sample.

17. The method of claim 16 wherein the affinity selection comprises immunoprecipitation of fragments that contain 5 methyl cytidine using an antibody to 5 methyl cytidine.

18. The method of claim 16 wherein the affinity selection comprises immunoprecipitation of fragments that contain 5 methyl cytidine using an antibody to a methyl binding protein.

19. The method of claim 16 wherein the affinity selection comprises immunoprecipitation of fragments that contain 5 methyl cytidine using an antibody to a protein that binds a methyl binding protein.

20. The method of claim 16 further comprising analysis of the second sample by hybridization to an array of probes attached to one or more solid supports, wherein said solid support is selected from the group consisting of a bead, a plurality of beads, one or more silica chips and one or more glass slides.
